(19) United States
(12) Patent Application Publication
(10) Pub. No.: US 2007/0161856 A1
(43) Pub. Date: Jul. 12, 2007

(54) Steering aid

(75) Inventor: Amir Belson, Sunnyvale, CA (US)

Correspondence Address: SHAY LAW GROUP LLP, 2755 CAMPUS DRIVE, SUITE 210, SAN MATEO, CA, 94403, US

(73) Assignee: NeoGuide Systems, Inc., Los Gatos, CA (US)

(21) Appl. No.: 11/603,944

(22) Filed: Nov. 21, 2006

Related U.S. Application Data
(60) Provisional application No. 60/739,248, filed on Nov. 23, 2005.

(51) Int. Cl. A61B 1/04; A61B 1/00

(52) U.S. Cl. 600/114

(57) ABSTRACT

The present invention provides embodiments of a steerable instrument having an elongate body having a distal end and a proximal end; a guide structure distal to the elongate body distal end; and a connector supported by the elongate body distal end and attached to the guide structure, wherein the guide structure or the connector is adapted to provide position feedback of the guide structure relative to the elongate body. There are also provided methods for advancing an instrument along a path by providing an indication of the direction of the path of the instrument by movement of a guide structure in response to interaction with an object defining the path; and adjusting the direction of the instrument based on the indication.

### CROSS-REFERENCE TO RELATED APPLICATIONS

This application claims the benefit of U.S. Provisional Application No. 60/739,248 filed Nov. 23, 2005, titled, “Steering Aid” which is incorporated herein by reference in its entirety.

### BACKGROUND OF THE INVENTION

Navigating medical instruments within the body is often a tedious process because of risk of harm to surrounding tissue and structures when instruments are misdirected. Moreover, movement of an instrument in a desired direction is compounded by narrow spaces, low light conditions and a general lack of clearly identifiable pathways. These factors and others make it difficult to accurately steer instruments within in the body.

Given the challenges presented in maneuvering instruments within the body, what are needed are improved devices and techniques to aid in steering those devices.

### SUMMARY OF THE INVENTION

One embodiment of the invention is a guide structure distal adapted to be part of, attached to or used in an existing channel of a steerable instrument having an elongate body with a connector supported by the elongate body distal end is attached to the guide structure. The guide structure or the connector is adapted to provide position feedback of the guide structure relative to the elongate body. In one embodiment, the steerable instrument is a guide tube. In one aspect, the connector and the guide structure extend along the longitudinal axis of the instrument. In another aspect, the guide structure has a generally spherical outer surface. In another aspect, the guide structure can be observed from a visualization device on the steerable instrument. In one alternative, a steering mechanism coupled to and controls the distal end of the steerable instrument. In one embodiment, the steering mechanism is under the control of an electronic motion controller that controls the movement of the elongate body. In one embodiment, the guide structure is hollow. In another, a coating on at least a portion of the guide structure to increase visibility of the guide structure. In one aspect, the coating on at least a portion of the guide structure is a reflective coating.

In one aspect, the guide structure has a stowed condition and a deployed condition. In another aspect, the guide structure is dimensioned to fit through the working channel of an instrument when in the stowed condition. In an alternative aspect, the guide structure is dimensioned to fit through a working channel of the steerable instrument.

In one embodiment, the position feedback of the guide structure relative to the elongate body is an electrical signal. In one aspect, deflection of the connector relative to the elongate body distal end provides position feedback of the guide structure relative to the elongate body. In another alternative, the position feedback of the guide structure relative to the elongate body is an input to an electronic motion controller used to control the steerable instrument. In another aspect, the deflection of a surface of the guide structure provides position feedback of the guide structure. In one embodiment, there is a sensor on or in the guide structure that generates a signal when bent. In one aspect, the signal generates an indication used to steer the elongate body. In another aspect, the signal is used to provide position feedback of the guide structure.

In one alternative embodiment, there is a roller on the guide structure. In another, the guide structure is an eversion tube. In yet another, the guide structure is adapted to be pneumatically advanced distally from the distal end of the elongate body distal end. In another aspect, the connector is a tether.

In one embodiment, there is provided a method for advancing an instrument along a path by providing an indication of the direction of the path of the instrument by movement of a guide structure in response to interaction with an object defining the path and then adjusting the direction of the instrument based on the indication. In one alternative, the adjusting step is performed by a user steering the instrument. In another alternative, the adjusting step is performed by a control system that manipulates the instrument. In one aspect, there is the step of looking though the guide structure to view the path. In one alternative, there is a step of pneumatically advancing the guide structure along the path. In another aspect, the indication of the direction of the path is provided by the movement of a connector attached to the guide structure. In one aspect, the movement of the guide structure in response to interaction with an object defining the path is deflection of the surface of the guide structure. In another aspect, the indication of the direction of the path of the instrument is provided through interaction of an instrument in the guide structure with the path. In yet another aspect, the indication of the direction of the path of the instrument is provided by a sensor or instrument that generates a signal when bent. In another aspect, the signal generated by the sensor provides bend information in two dimensions.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 illustrates an embodiment of a steering aid having a guide structure on a deformable connector.

FIG. 2 illustrates another embodiment of a steering aid having a touch pad sensor positioned to indicate the movement of the guide structure.

FIG. 3 illustrates another embodiment of a steering aid having a ball/socket sensor adapted to indicate the movement of the guide structure.

FIGS. 4, 4A and 4B illustrate an embodiment of the present invention that integrates a spring with or without instrumentation into an embodiment of a guide structure.

FIG. 5 illustrates the incorporation of a strain gauge into a guide structure.

FIG. 6 illustrates the incorporation of a bimetallic strip adapted to indicate the deflection of the guide structure.

FIG. 7 illustrates a ribbed guide structure with semi-solid features.

FIG. 8 illustrates a ribbed guide structure.

FIG. 9 illustrates an instrumented deformable guide having the instrumentation enclosed within a solid guide structure having an outer skin.

FIG. 10 illustrates the distal end of an instrument that may be adapted to hold, receive or engage with a guide structure embodiment of the present invention.

FIG. 11 illustrates a ball shaped guide structure distal of a semi-rigid stem.

FIG. 12 illustrates a ball shaped guide having treads or reversion rollers along the sides to engage a wall.

FIG. 13 illustrates a ball shaped guide having wheels or rollers along the sides to engage a wall.

FIG. 14 illustrates a parachute style guide structure.

FIG. 15 illustrates an eversion tube or other structure suited to extension when air is applied.

FIG. 16 illustrates the embodiment of FIG. 15 after air is introduced thereby forming a guiding structure for the instrument to advance along.

FIG. 17 illustrates an embodiment of a steering aid on an exemplary guide tube.

FIG. 18 illustrates an embodiment of a steering aid on another exemplary guide tube.

### DETAILED DESCRIPTION

There are a number of steerable instruments available. Examples of steerable instruments, including without limitation guide tubes and rigidizable guide tubes and various control systems are described in, for example, U.S. Pat. Nos. 6,468,203 and 6,858,008 and U.S. application Ser. No. 10/988,212, filed Nov. 12, 2004, entitled “Articulating Connector Device for Endoscopes,” U.S. Pat. No. 6,800,056; U.S. Pat. No. 6,837,846 and Published U.S. Patent Application US 2003-0171650 published Sep. 11, 2003, each of which is incorporated herein by reference in its entirety. The applications and patents listed above are commonly assigned with this application.

Embodiments of the present invention relate to steering aids that assist a user to steer an instrument. The instrument may be an endoscope or colonoscope, for example, or a guide to steer another instrument. A passive or an active steering aid may be used to assist in manual and/or automatic steering of an instrument while traversing a portion of the body. Active steering aids provide a control system input for automatic control and navigation of an instrument. Passive steering aids provide an indication of the proper or desired direction in a lumen to assist in the manual steering control of an instrument. Combination steering aids having aspects of active and passive systems are also possible.

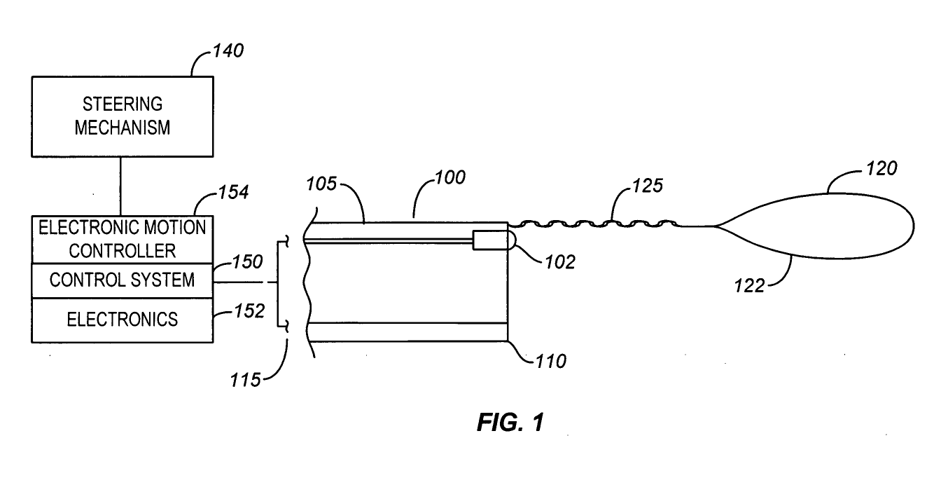

A passive guide system refers to a steering aid system that provides information only. The information is typically in the form of a visual cue to a user. Such as the reflection off of the guide structure or some other indication from the guide structure about the direction to maneuver the instrument. Consider the example in FIG. 1 where an optional reflective coating 122 is added to the guide structure 120. Generally, user action is needed in a passive guide system in order for the steerable instrument to adjust direction in response to the information or indications provide by the steering aid system.

In contrast, an active system is configured such that the information provided by the steering aid system is provided into and used by the control system for the steerable instrument. Hybrid guide systems are also possible where the information provided by the steering aid system is displayed or communicated to a user for user initiated response to control the steerable instrument as well as input to the guidance or control systems for the steerable instrument for use in those systems as well.

FIG. 1 illustrates an embodiment of a passive steering aid. Many of the general characteristics of steering aid embodiments of the invention are described in relation to FIG. 1. This explanation is for clarity of the general characteristics and operation of steering aid embodiments of the invention. This steering aid embodiment includes a steerable instrument 100, a deformable connector 125, and a guide structure 120. The steerable instrument 100 may be any device that can be inserted into the body, such as, for example, an endoscope, a rigidizable guide tube, or a colonoscope. The guide structure 120 and connector 125 may be integrally formed as components of the steerable instrument or provided as separate components to be mounted on a separate steerable instrument. Additionally, the guide structure 120 and connector 125 may be configured to extend out through an instrument working channel as a stand alone device or may be integrated into a working channel or dedicated channel of an instrument.

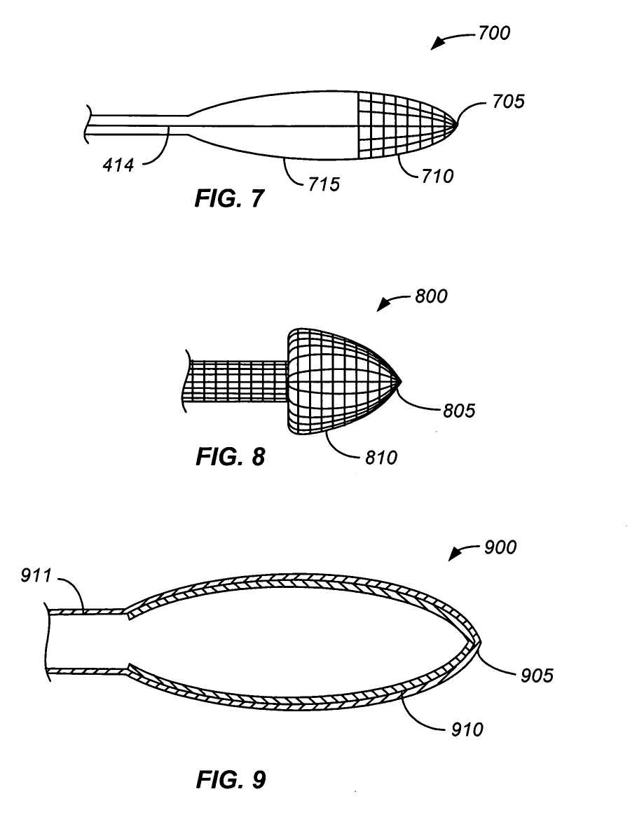

Although the steerable instrument of the present invention has been described for use as a colonoscope, the instrument can be configured for a number of other medical and industrial applications. In addition, the present invention can also be configured as a catheter, cannula, surgical instrument or introducer sheath that uses the principles of the invention for navigating through tortuous body channels. The guide structure may be comprised of a balloon, deformable structure, or other structure that is atraumatic when in contact with the surrounding objects, tissue or structures. The guide structure 120 may be hollow, solid, semi-solid (FIG. 7), or an open structure (FIG. 8 and FIG. 11). The deformable connector 125 may be any suitable flexible structure. In one embodiment, the connector is a spring-like device as shown in FIG. 1. The deformable connector 125 is rigid enough to maintain the guide structure 120 in a distal most relation to the steerable instrument distal end 110. The deformable connector 125 may also be configured so that it will not loop during operation. The deformable connector 125 is rigid enough to support the weight of the guide structure 120 but is not so rigid as to not allow the guide structure 120 to deflect when placed in contact with surrounding objects, tissue or structure. It is to be appreciated that the deformable connector and guide structure combination is suitably adapted to accommodate the portion of the body that the steerable instrument will navigate.

In an illustrative embodiment where the deformable connector and guide structure are intended for use in the colon, then the rigidity, shape, size, materials and other qualities and characteristics of the design and operation of the deformable connector and guide structure are selected such that when the guide structure is urged against the colon wall, the deformable connector will deflect to maintain atraumatic contact of the guide structure with the colon wall. In other alternatives embodiments, the rigidity, shape, size, materials and other qualities and characteristics of the design and operation of the deformable connector and guide structure are adapted to accommodate other hollow body organs, lumens or pathways as desired for a particular application.

FIG. 1 illustrates a steerable instrument 100 having an elongate body 105 with a distal end 110 and a proximal end 115. A guide structure 120 is positioned distal to the elongate body distal end 110. Optionally, the guide structure may include have a coating 122 on at least a portion of the guide structure to increase visibility of the guide structure. The coating 122 may be a reflective coating to aid in visual identification of the guide structure.

A connector 125 supported by the elongate body distal end 110 is attached to the guide structure 120. The guide structure 120 or the connector 125 is adapted to provide position feedback of the guide structure 120 relative to the elongate body 105. As illustrated, the connector 125 and the guide structure 120 extend along the longitudinal axis of the instrument 100. FIG. 1 also illustrates that guide structure 120 is positioned so that it can be observed from a visualization device 102 on the steerable instrument. The visualization device 102 may be any conventional endoscopic lighting, imaging, or video device. Also illustrated in FIG. 1 is a steering mechanism 140 that coupled using conventional techniques to the distal end of the steerable instrument 100. In one embodiment, the steering mechanism 140 is under the control of an electronic motion controller 154 that controls the movement of the elongate body 105 and the steerable instrument 100.

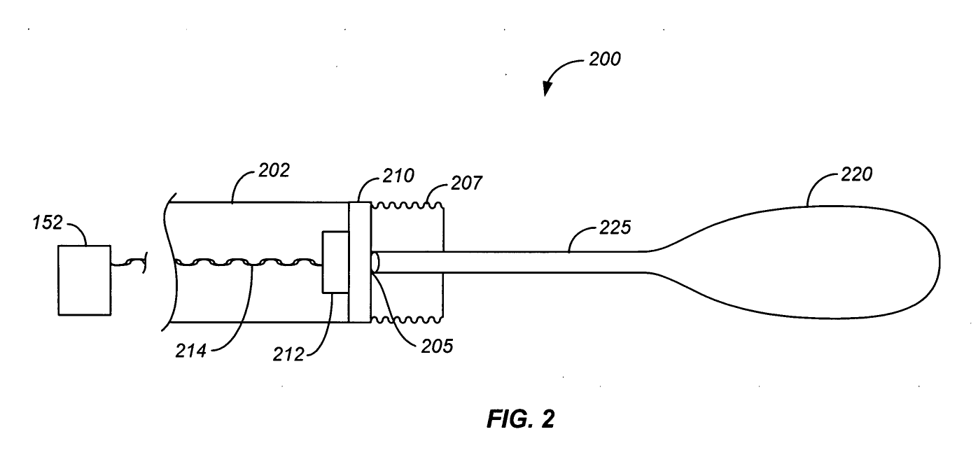

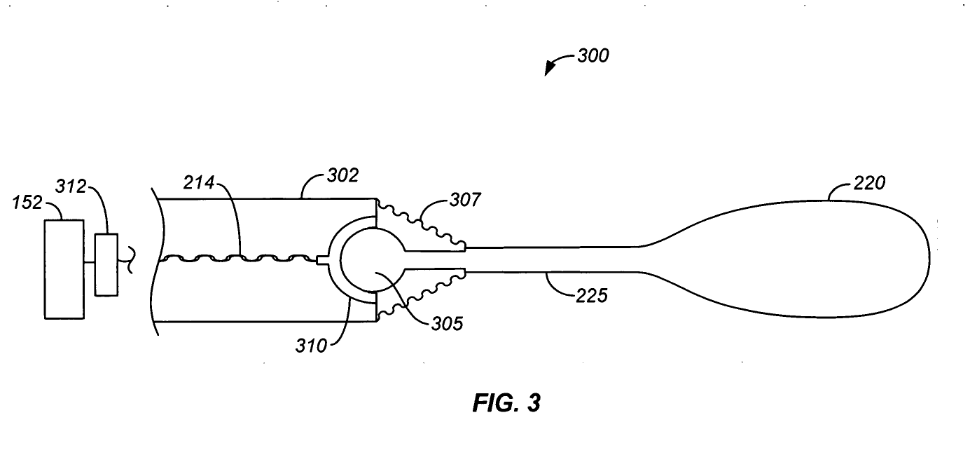

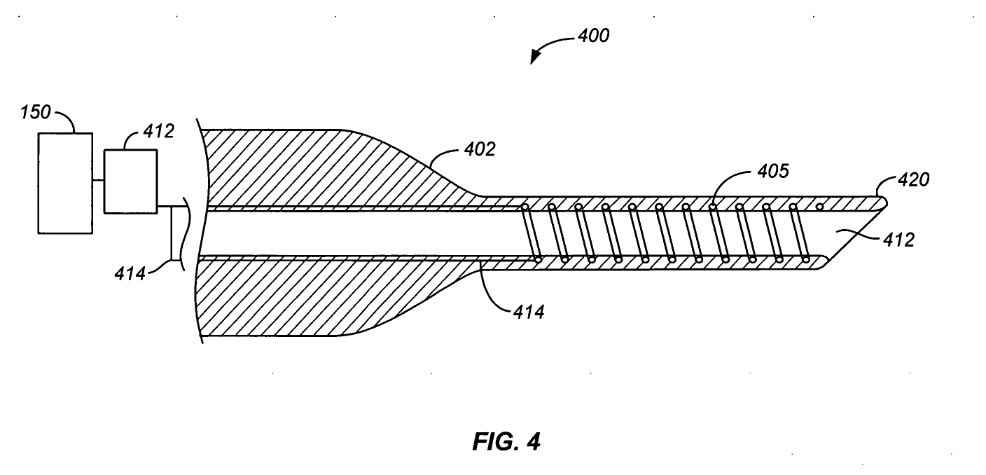

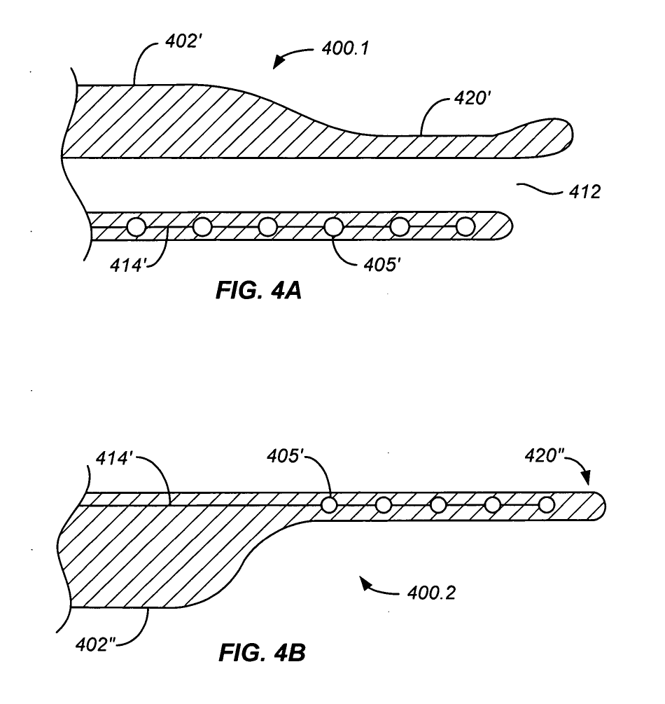

FIG. 1 also illustrates a conventional control system 150 having electronics 152 suited to the operation of the steerable instrument and the various components of the steering aids of the invention. The electronics 152 vary depending upon the complexity of the steering aid as will be appreciated by those of ordinary skill in the art. The electronics used to interpret signals from the steering aids in FIGS. 2, 3, for example, will be different from those needed to interpret sensor and/or instrument signals from the steering aids in FIGS. 4-9. Electronics will also be modified to accommodate different signal types such as electrical, optical and magnetic to name a few. It is to be appreciated that the electronics 152 include conventional electronic components to interpret signals from the guide structure or other components used in the steering aid. Electronics 152 may also be used to provide outputs or indications from the guide structure such as visual outputs, video outputs, audio outputs or steering direction indications and the like.

The functionality of the deformable connector may change depending upon the operational characteristics of the steering aid system. In one embodiment, the deformable connector is simply the structure that extends the guide structure a distance from the distal tip of the steerable instrument (FIG. 1.) The movement of guide structure as it contacts and is deflected from the lumen wall is observed using conventional endoscopic video or optics systems. The steerable instrument is then directed by a user based on the observed or indicated movement of the guide structure. In an alternative embodiment, the movement of the deformable connector in response to the movement of the guide structure is connected to or provides an indication of guide structure movement (FIGS. 2 or 3). It is to be appreciated that the steering aid also provides a signal when the guide is straight (not bent or deflected) when that position indicates the path or lumen position. In these and other embodiments described herein, suitable control electronics are connected via wires or wirelessly to receive signals from the sensors, instruments, instrumented deformable member or other components of a steering aid of the invention. In another alternative, the deformable connector and guide structure combination utilize the deformable connector for both types of indications.

Examples of the kinds of indications or steering aid provided by embodiments of the present invention:
a) A monitor may be used to display to a user the guide structure's path that the steerable instrument should follow.
b) There may be a graphical indication on a display of the path that the steerable instrument should follow.
c) A graphical indication (such as from (b) above) with feedback indications of steering corrections or correctness of the current course. The feedback indications of steering corrections or correctness may be accomplished by visual means, for example using colors such as red, yellow, green or text messages or other feedback techniques to indicate a manner for the user to guide the steerable instruments to follow the guide structure.
d) Audible means may also be used to indicate to a user the path to follow. Said audible means can be, for example, a system or series of beeps or voice commands.
e) The various feedback techniques in (b),(c) or (d) above may be used in combination or with other feedback systems such as hepatic feed back or others to provide perceptible indication to a user for directing the steerable instrument.

It is to be appreciated that signals can be sent from the steering aid device (the guide structure and/or deformable connector) into a control system for automated control of the steerable instrument. The signals will vary based on the type of instruments or systems used to monitor or detect the movement of the guide structure or other components in the steering aid. These signals may be provided by any number of conventional systems that are based on magnetic, electromagnetic, optical, mechanical, and/or electromechanical sensors and systems.

Any of the above indicators are fed into, captured by, utilized with, or used to modify, or are otherwise used in conjunction with an electronic map of the lumen being traversed. The steering aid may be used to record the shape of the lumen at a specific time, depth, or position and such information can then be sent to the electronic map of the lumen.

The guide structure is attached to a deformable connector that is attached to a steerable instrument as illustrated in FIG. 1. The steering aid system could be augmented to further assist in the identification of the lumen pathway through the use of conventional medical imaging techniques or other techniques such as echograms, ultrasound, optical sensor systems using non-visable light spectrum, lasers, visible light, or other spectrums of light. In one embodiment a transmitter/receiver is provided on the steerable instrument in communication with the guide structure. The guide structure is made from, coated with or otherwise provides a surface or features that is identifiable to or provides a return signal to the transmitter/receiver. The position, deflection and/or shape information from the steering aid could be combined with an depth measurement indicating the position of the steering aid. The combination of the steering aid bend or position with depth could be used to generate a map of the path or lumen being following by the steering aid.

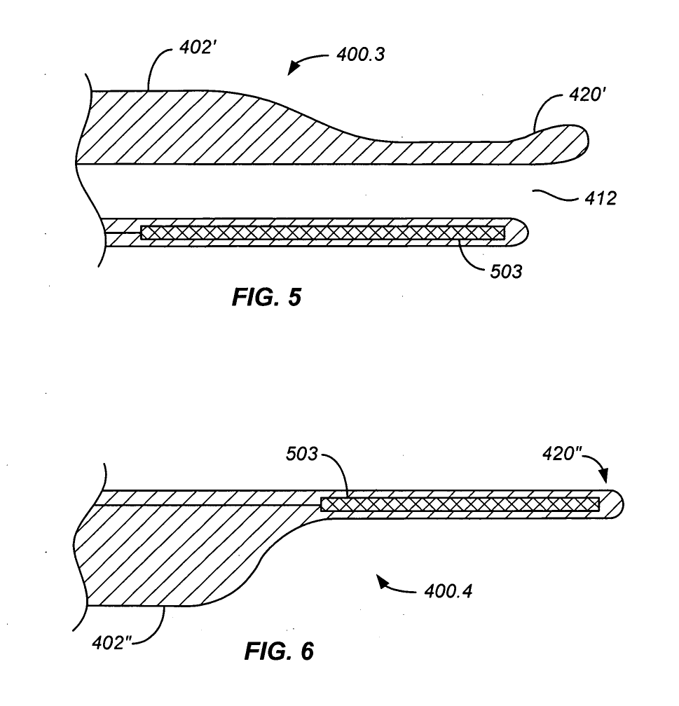

The guide structure may have any of a variety of shapes such as a simple (e.g., FIG. 1) or compound (e.g., FIGS. 5, 6, or 8) shapes. The guide structure may have circular, elliptical, rectangular, and polygonal or hybrid cross sections. The guide structure may have an elongate body with a round tip as illustrated in FIG. 1 or other shape suited to the lumen of body pathway being traversed. The guide structure may be solid or may be a non-solid (i.e., ribbed only as in FIG. 8) or semi-solid (i.e., ribbed structure with one or more solid features as in FIG. 7).

In addition, the guide structure may be a deformable structure having suitable instrumentation such as strain gauges, micrometers, or other suitable measurements devices within or attached to the guide structure (see, e.g., FIGS. 5-9).

In one illustrative embodiment, the guide structure is a mesh with instrumentation coupled to the mesh to allow indication of mesh deformation (FIGS. 7 or 8). The deformation of the mesh is correlated to contact with a portion of the lumen or pathway. The correlated contact is then provided to a user to aid in steering. In general operation, the deformation of the guide structure is measured, correlated to contact and processed to be useful to a user. The processed measurement and the resulting processed information is then communicated to a user for manual control of the steering instrument, as an automatic input into a control system, as part of a feedback system to either manual or automatic control or combinations thereof. The deformation of the guide structure is communicated to the user to provide an indication of contact with or the direction of the body lumen or pathway.

FIG. 2 illustrates another alternative a steering aid 200 of the invention. In this embodiment, the proximal end of the connector 225 has a tip 205 adapted to interact with a touch sensitive pad 210. Bellows 202 protects the touch pad and tip 205; The movement of the tip 205 on the touch pad 210 is related to the deflection of the guide structure in response to interaction with an object. The movement of the tip 205 in relation to the touch pad 210 is interpreted by touch pad electronics 212. Electronics 212 are connected to the system controller 152 using suitable wiring 214. In use, the guide structure 220 moves or deflects when in contact with tissue or structures. This causes the shaft tip 205 to move or deflect against the touch pad 210. The touch pad electronics 212 interpret the signal and communicate with the control system 152. The touch pad signals can then be used by the system controller to provide position information to a user or for use in controlling a steerable instrument.

FIG. 2 illustrates a guide structure 200 positioned distal to an elongate body distal end. A connector 225 is attached to the guide structure 220 and supported by the elongate body distal end (i.e., attached to the distal end as in FIG. 1 or through a working channel). The guide structure or the connector is adapted to provide position feedback of the guide structure relative to the elongate body. In one embodiment, the guide structure 200 is dimensioned to fit through a working channel of the steerable instrument. In another embodiment, the guide structure 220 is formed as part of the steerable instrument as in FIG. 1. In another embodiment, the guide structure is dimensioned to fit through a new channel or additional channel added to the instrument to provide guide structure access.

FIG. 3 illustrates another steering aid embodiment of the present invention. In this embodiment, the movement of the guide structure 220 is translated using the movement of the ball 305 relative to the socket 310 to indicate or mimic guide structure 220 movement. The movement of ball 305 relative to the socket 310 is interpreted using electronics 312 and communicated to the control system 152. As will all embodiments described herein, the electronic components used to interpret guide structure signals are illustrated separately for purposes of discussion. As is conventional in many control systems, all electronics for the system may be provided in a single integrated system controller.

In this embodiment, the proximal end of the connector 225 has a tip 305 adapted to interact with a touch sensitive socket 310. Bellows 207 protects the socket 310 and tip 305. The movement of the tip 305 on the socket 310 is related to the deflection of the guide structure 220 in response to interaction with an object. The movement of the tip 305 in relation to the socket 310 is interpreted by socket electronics 312. Electronics 312 are connected to the socket 310 and the system controller 152 using suitable wiring 214. In use, the guide structure 220 moves or deflects when in contact with tissue or structures. This causes the socket 305 to move or deflect relative to socket 310. The touch pad electronics 312 interpret the signal and communicate with the control system 152. The socket signals can then be used by the system controller to provide position information to a user or for use in controlling a steerable instrument.

FIG. 3 illustrates a guide structure 300 positioned distal to an elongate body distal end. A connector 225 is attached to the guide structure 220 and supported by the elongate body distal end (i.e., attached to the distal end as in FIG. 1 or through a working channel). The guide structure or the connector is adapted to provide position feedback of the guide structure relative to the elongate body. In one embodiment, the steering aid 300 is dimensioned to fit through a working channel of the steerable instrument. In another embodiment, the guide structure 220 is formed as part of the steerable instrument as in FIG. 1.

FIGS. 4, 4A and 4B illustrate steering aid embodiments that integrate an instrumented deformable member into the guide structure. Deflection of the guide structure is detected, measured, or otherwise indicated by the instrumented deformable member. The signals indicate deflection of the guide structure. The guide structure deflection indicates lumen pathway, said indication of lumen pathway is used to steer the steerable instrument as described above. The instrumented deformable member may be integrated into the steerable instrument and have one or more lumens (FIG. 4), extended only along one side (i.e., not include a central lumen as in FIG. 4A) or be part of an instrumented deformable tip (FIG. 4B).

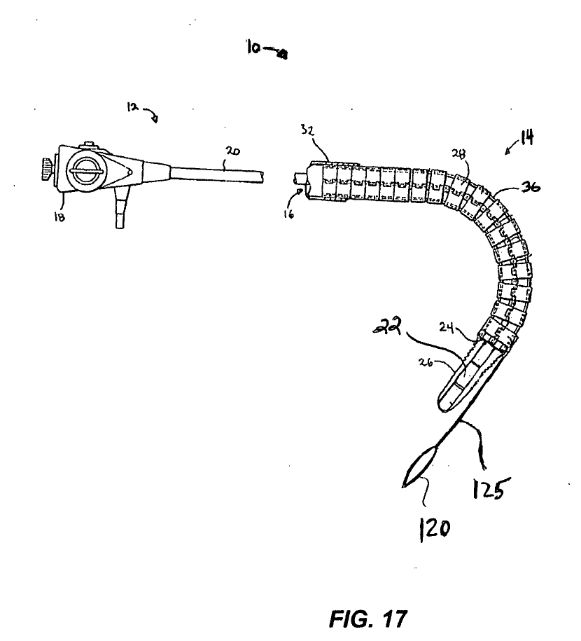

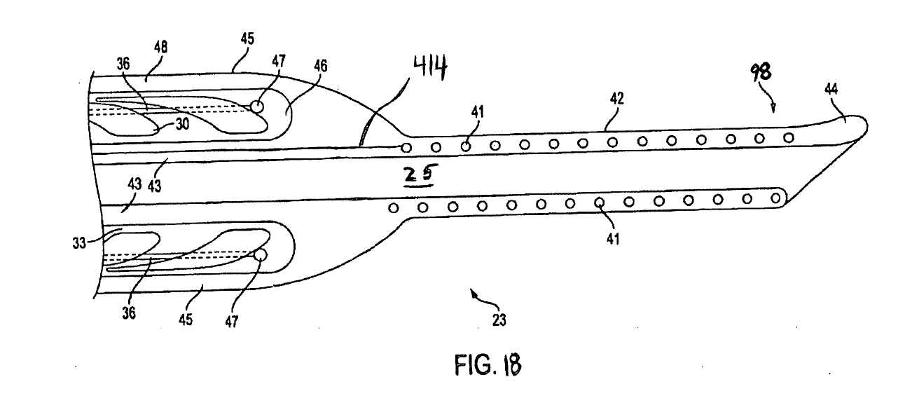

FIG. 4 illustrates a steering aid 400 having an instrumented spring 405 positioned within a tip 420 of a body 402. An optional lumen 412 sided to provide access for a steerable instrument is provided. Rigidizing elements may also be included as described below in FIGS. 17 and 18. Wiring 414 connects the spring 405 to electronics 412 and control system 150. FIG. 4A illustrates an embodiment of a steering aid 400.1 having an instrument or sensor 405′ connected to suitable electronics with wiring 414′. The tip 420′ extends from the body 402′ with a lumen 412 extending there through. The instrument 405′ is connected to suitable electronics using wiring 414′. FIG. 4B illustrates a steering aid 400.2 that has an instrument 405′ in tip 420″. The steering aid 400.2 has a body 402″ dimensioned to fit within the working channel of an instrument. The instrument 405′ is connected to suitable electronics using wiring 414′.

In these embodiments, the instrumented deformable member may utilize a spring having movement that is correlated to the deflection of the guide structure. The correlated deflection of the spring can be used as described herein to indicate a pathway to follow. While illustrated as a spring 405 that can detect deflection in these exemplary embodiments, it is to be appreciated that the instrumented deformable tip may be selected from any of a wide variety of instruments suited to measuring deflection such as strain gauges or other instruments known to those of ordinary skill. For example, a bend sensor (Flexpoint Sensor Systems, Inc. Draper, Utah) or any instrument or sensor that generates a signal when bent or deflected may be used. Once the sensor is moved by the guide structure or other component of the steering aid, the movement of the sensor is correlated to the position relative to the distal end of the elongate body.

Sensors or instruments may be printed or fabricated onto flexible substrates and embedded into the body of the guide structure as illustrated herein. Several sensors may be used in combination and/or with different orientation to provide two dimensional bend or deflection information. The two dimensional bend or deflection information may then be output as steering commands to a control system or as an output to a user (i.e., degree of left or right steering combined with degree of up or down steering, for example). One dimensional or three dimensional outputs and steering indications may also be provided depending upon application.

FIG. 5 illustrates a guide structure 400.3 on the end of a body 402′. FIG. 5 also illustrates the incorporation of a strain gauge or other sensor 503 that produces a signal when deflected into the tip 420′ of the guide structure 400.3. The signal produced by sensors 503 are interpreted as described above with regard to FIGS. 2 and 3.

It is to be appreciated that the steering aids 400, 400.1, 400.3 of FIGS. 4, 4A and 5 may also be adapted dimensioned to fit through a working channel of the steerable instrument. This size of steering aid may be accomplished by removing or restricting the size of the optional lumen 412. With reference to FIG. 4, in the newly dimensioned steering aid 400, the sensor 405 would extend along and within the tip 420 (lumen 412 is filled in so that tip 420 is solid and of smaller diameter). With reference to FIG. 4A, in the newly dimensioned steering aid 400.1, the sensor 405′ is within tip 420′ when lumen 412 is removed or filled in. With reference to FIG. 5, in the newly dimensioned steering aid 400.3, the bend sensor 503 is within tip 420′ when lumen 412 is removed or filled in.

In FIG. 6 the sensor 503 incorporated into the tip 420″ is, for example, a bimetallic strip adapted to indicate the deflection of the guide structure 400.4.

FIG. 7 illustrates another alternative a steering aid 700 of the invention. FIG. 7 illustrates a guide structure 705 positioned distal to an elongate body distal end. The guide structure 705 has a see through mesh portion 710 that may comprise the entire structure or, optionally, a solid portion 715 may be provided. A connector 722 is attached to the guide structure 705 and supported by the elongate body distal end. The guide structure or the connector is adapted to provide position feedback of the guide structure relative to the elongate body. The position feedback may be provided by signals generated in response to the mesh portion 710 of the guide structure 705 and communicated to suitable electronics via wiring 414. In one embodiment, the guide structure 705 has a stowed condition and a deployed condition. The guide structure 705 is illustrated in the deployed condition in FIG. 7. When in the stowed condition, the guide structure is dimensioned to fit through a working channel of the steerable instrument. Alternatively, the steering aid 700 may be formed as part of the instrument as in FIG. 1.

FIG. 8 illustrates another alternative a steering aid 800 of the invention. FIG. 8 illustrates a guide structure 805 positioned distal to an elongate body distal end. The guide structure 805 has a flared tip made entirely of a see through mesh portion 810. A connector 822 is attached to the guide structure 705 and supported by the elongate body distal end. The guide structure or the connector is adapted to provide position feedback of the guide structure relative to the elongate body. The position feedback may be provided by signals generated in response to the mesh portion 710 of the guide structure 705 and communicated to suitable electronics as discussed above. In one embodiment, the guide structure 805 has a stowed condition and a deployed condition. The guide structure 805 is illustrated in the deployed condition in FIG. 8. When in the stowed condition, the guide structure is dimensioned to fit through a working channel of the steerable instrument. Alternatively, the steering aid 800 may be formed as part of the instrument as in FIG. 1.

FIG. 9 illustrates an instrumented deformable guide 900 having the instrumentation 910 enclosed within a solid guide structure having an outer skin 905. As illustrated in the embodiment of FIG. 9, the instrumented deformable guide 900 may also have the instrumentation enclosed within a solid guide structure having an outer skin that is deformable in response to contact with the lumen or pathway. The instrumentation 910 within the outer skin 905 is adapted and configured to measure or otherwise indicate the deformation of the outer skin. It is to be appreciated that the instrumented deformable guide structure may be used alone or in combination with a deformable connector embodiment and/or the passive and active guide systems described herein.

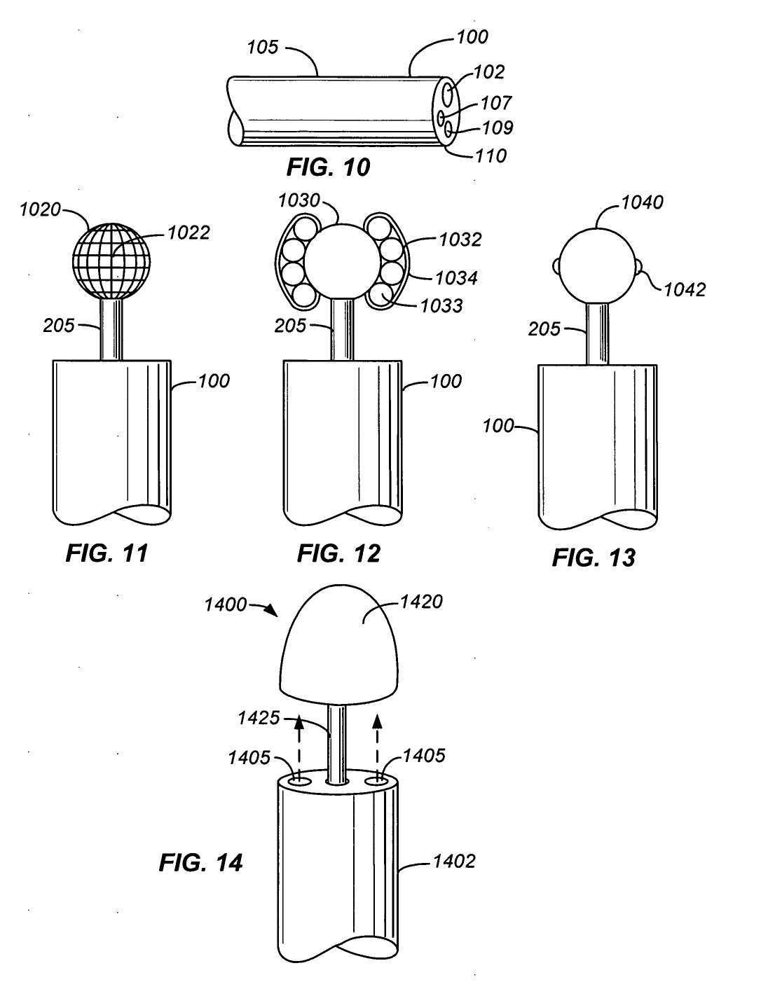

FIG. 10 illustrates the distal end of an instrument having conventional lighting or imaging means 102 and two working channels 107, 109. Other channels may also be provided as needed. The working channels 107, 109 or the instrument 100 may be adapted to hold, receive or engage with a guide structure embodiment of the present invention. Guide structures 1020, 1030 and 1040 are illustrative of guide structure that may be (a) stowed into a compact configuration (not shown), (b) passed along through an instrument working channel and (c) placed into a deployed condition for use as shown in FIGS. 11, 12 and 13.

The guide structures 1020, 1030 and 1040 in FIGS. 11, 12 and 13, respectively, have a generally spherical outer surface. Each of these steering aids is shown distal to the distal portion of a steerable instrument 100. FIG. 11 illustrates an open framed ball shaped guide structure 1020 distal of a semi-rigid connector 205. FIGS. 12 and 13 also illustrate guide structures having rollers. FIG. 12 illustrates a ball shaped guide 1030 having treads or eversion rollers along the sides to engage a wall or object. FIG. 12 illustrates a tracked assembly 1032 positioned on the exterior of the guide structure 1030. The track assembly 1032 includes a tread 1034 around a plurality of rollers 1033. When the guide structure 1030 encounters an object with track 1034, the track 1034 slides over the rollers 1033 so that the guide structure 1030 may advance beyond the object. FIG. 13 illustrates a ball shaped guide 1040 having wheels or rollers 1042 along the sides or on its outer surface for the same reasons.

FIG. 14 illustrates a parachute style guide structure adapted to be pneumatically advanced distally from the distal end of the elongate body distal end. In this steering aid embodiment, air (provided behind the parachute 1420 from nozzles 1405) advances the parachute 1420 along the lumen. In order to reduce drag, the connector that attaches the parachute guide structure 1420 is a tether 1425. When air is injected into the parachute 1420, the parachute 1420 opens into the lumen making it easy to see. Continued application of air then propels the parachute 1420 along the lumen. A user watches and follow the advancement of the parachute 1420 to navigate the lumen. This embodiment provides a method of pneumatically advancing the guide structure along a path.

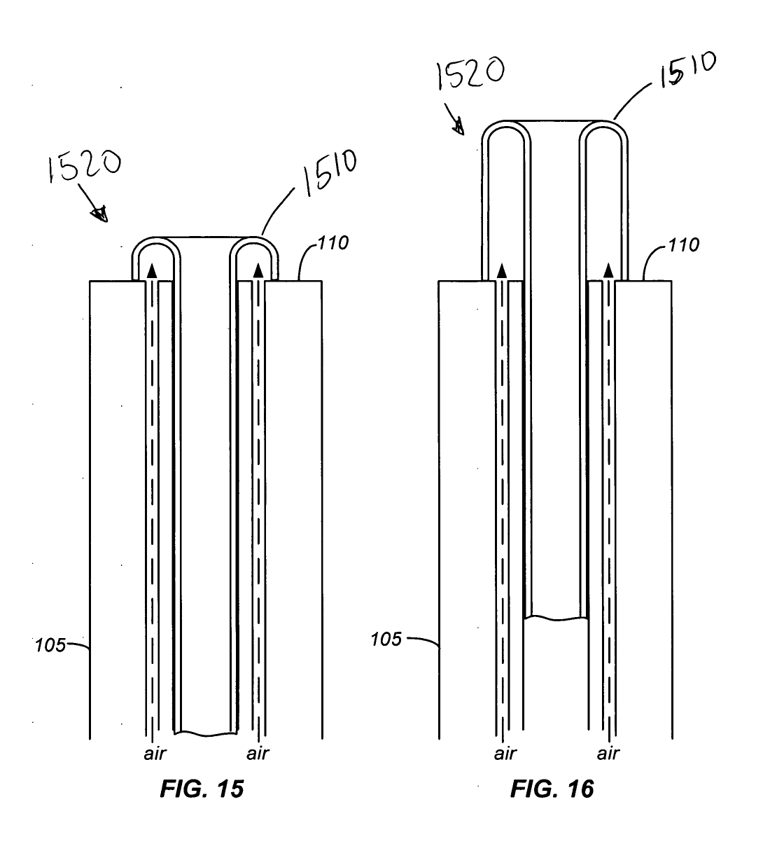

FIGS. 15 and 16 illustrate another steering aid having a guide structure that is adapted to be pneumatically advanced distally from the distal end of the elongate body distal end. The guide structure in this embodiment of the invention is a thin walled everted structure suited to extension when air is applied as illustrated. In the illustrative embodiment of FIG. 15, the guide structure 1520 includes an eversion tube 1510. FIG. 16 illustrates the embodiment of FIG. 15 after air is introduced thereby forming a guiding structure for the instrument to advance along. In use, as air is introduced into the guide structure 1520, it advances from a position adjacent the distal end 110 as shown in FIG. 15 to a position away from the distal end 110 as shown in FIG. 16. Additional details regarding the construction and use of eversion tubes may be found in U.S. Pat. No. 6,358,199 which is incorporated herein by reference in its entirety. This is an additional embodiment that provides a method of pneumatically advancing the guide structure along a path.

FIG. 17 illustrates an embodiment of a guide tube adapted to use a steering aid according to the invention. The guide tube assembly 10 is seen partially disassembled for clarity. Assembly 10 generally comprises an endoscope 12 which is insertable within guide tube 14 through the guide lumen 16. The guide tube comprises a plurality of segments 28 that are selectively rigidizable using tensioning elements 36. The guide tube is covered by a flexible covering 32. The guide tube 14 has been modified to accommodate a steering aid 120. The guide structure 120 is attached to the connector 125. The connector 125 is attached to the guide tube near distal opening 24. The guide tube may be equipped with a steering mechanism, steering means for a user to steer and visualization means to allow a user to see the guide structure and the guide tube surroundings. Alternatively, the steering and visualization system and controls of the endoscope 12 may be used to see the guide structure 120 and manipulate the guide tube accordingly (i.e., using the steering system of the endoscope or a steering system adapted to steer the guide tube). Endoscope 12 may be any conventional type endoscope having a handle 18 with a shaft 20 extending therefrom. The distal end of shaft 20 includes a controllable distal portion 22 which may be manipulated to facilitate steering of the device through the body. Endoscope shaft 20 may be slidingly disposed within the guide lumen 16 such that the controllable distal portion 22 is able to be passed entirely through guide tube 14 and out distal opening 24 defined at the distal end of tube 14. Additional details of the guide tube 14 are provided in U.S. Pat. No. 6,837,846, incorporated herein by reference in its entirety. Guide tube 14 may be modified to accommodate any of the steering aid embodiments described herein. The guides described in U.S. Pat. No. 6,800,056 may also be modified to include a steering aid of the invention. U.S. Pat. No. 6,800,056 is incorporated herein by reference in its entirety and is, like U.S. Pat. No. 6,837,846, commonly assigned to the assignee of this application.

FIG. 18 illustrates the distal region 23 and atraumatic tip 98 of an embodiment of steering aid 400 adapted for use with a guide tube. Distal region 23 comprises an instrument or sensor 41 to detect the deflection of the tip 24 and provide a signal related to the deflection. That signal is then used to provide an indication to a user on how to steer the elongate body distal tip. The signal may also be provided to an electronic motion controller. Instrument 41 is encapsulated in flexible layer 42 and is connected to suitable electronics via wiring 414. Layer 42 preferably comprises a soft elastomeric and hydrophilic coated material, such as silicon or synthetic rubber, and terminates at the distal end in enlarged section 44 that forms atraumatic tip 24. At the proximal end, layer 42 joins with or is integrally formed with liner 43 that extends through bores 33 of nestable elements 30 to a proximal handle (not shown). Tensioning elements 36 are used to lock the nestable element 30 into position. Liner 43 is made of a thin, flexible material optionally having flexible, kink-resistant coil (not shown) embedded therein. Layer 42 preferably joins with or is integrally formed with flexible elastomeric skin 45 to form sheath 48, which encapsulates nestable elements 30 in annular chamber 46. Skin 45 provides a relatively smooth outer surface for overtube 22, and prevents tissue from being captured or pinched during relative rotation of adjacent nestable elements 30. An optional lumen 25 is provided to allow the passage of instruments through the guide tube. Alternatively, the lumen 25 could be removed and the guide tube dimensioned to fit through a working channel of a steerable instrument such as a colonoscope, endoscope and the like. In use, the tip 24 interacts with an object. The resulting deflection of the tip 24 is detected by the sensor or instrument 41 (i.e., a bend sensor, a strain gauge, a spring, for example) and provided to the user. Additional details of the guide tube are available in U.S. Pat. No. 6,942,613 incorporated herein by reference in its entirety. Additionally, various shapes of the guide structure include those listed in U.S. Pat. No. 6,942,613. The shapes also include those shapes listed in the NeoGuide patents and patent applications described above, which are incorporated by reference herein. The shape may also depend on the lumen and/or the physiology of the anatomy to be traversed.

The various steering aid embodiments described herein enable numerous improved methods of guiding instruments or maneuvering along pathways or lumens. In one embodiment there is provided a method for advancing an instrument along a path by providing an indication of the direction of the path of the instrument by movement of a guide structure in response to interaction with an object defining the path. Next, adjusting the direction of the instrument based on the indication. The adjusting step may be performed by a user steering the instrument, such as through the use of the steering mechanism 140 in FIG. 1. In addition to this manual process, the actions of the user may be aided by the use of a system controller 150 alone or in combination with an electronic motion controller 154 as is described in U.S. Pat. Nos. 6,468,203 and 6,858,008. As such, the adjusting step is performed by a control system that manipulates the instrument. Signals generated by the steering aid in response to interaction with objects may also be input into the control system or electronic motion controller.

The indication of the direction of the path of the instrument may be provided in a number of ways by various embodiments of the invention. In one embodiment, the indication of the path is provided through interaction of an instrument in the guide structure with the path. Examples of this method are described above in FIGS. 4-6 and 9, for example. Alternatively, the indication of the direction of the path is provided by the movement of a connector attached to the guide structure. Examples of this method are described above in FIGS. 1-3, and 11-13, for example. More specifically, the movement of the guide structure in response to interaction with an object defining the path is deflection of the surface of the guide structure. Examples of this method are described above in FIGS. 7-9 for example. Alternatively, the indication of the direction of the path of the instrument is provided by a sensor or instrument that generates a signal when bent. In one embodiment, the signal generated by the sensor provides bend information in two dimensions.

The method of steering the instrument using the steering aid may also include the ability to get position information from the steering aid but also look through the steering aid. This step is provided by embodiments such as steering aids 700, 800, and 1020 for example. In this way, the method also includes looking though the guide structure to view the path.

While numerous embodiments of the present invention have been shown and described herein, one of ordinary skill in the art will appreciate that such embodiments are provided by way of example only. Numerous variations, changes, and substitutions will now occur to those skilled in the art without departing from the invention. In addition, the intended uses of the present invention include a variety of medical applications, it is to be appreciated that the instrument controlling techniques described herein have industrial applicability as well. It should be understood that various alternatives to these embodiments of the invention described herein may be employed in practicing the invention. It is intended at the following claims defined the scope of the invention and it methods and structures within the scope of these claims and their equivalents be covered thereby.

1. A steerable instrument, comprising:
an elongate body having a distal end and a proximal end;
a guide structure distal to the elongate body distal end; and
a connector supported by the elongate body distal end and attached to the guide structure, wherein the guide structure or the connector is adapted to provide position feedback of the guide structure relative to the elongate body.

2. The steerable instrument of claim 1 wherein the connector and the guide structure extend along the longitudinal axis of the instrument.

3. The steerable instrument of claim 1 wherein the guide structure has a generally spherical outer surface.

4. The steerable instrument of claim 1 wherein the guide structure can be observed from a visualization device on the steerable instrument.

5. The steerable instrument of claim 1 further comprising:
a steering mechanism coupled to the distal end of the steerable instrument.

6. The steerable instrument of claim 5 wherein the steering mechanism is under the control of an electronic motion controller that controls the movement of the elongate body.

7. The steerable instrument of claim 1 wherein the position feedback of the guide structure relative to the elongate body is an electrical signal.

8. The steerable instrument of claim 1 wherein the guide structure is hollow.

9. The steerable instrument according to claim 1 further comprising:
a coating on at least a portion of the guide structure to increase visibility of the guide structure.

10. The steerable instrument according to claim 9 wherein the coating on at least a portion of the guide structure is a reflective coating.

11. The steerable instrument according to claim 1 wherein the guide structure has a stowed condition and a deployed condition.

12. The steerable instrument according to claim 11 wherein the guide structure is dimensioned to fit through a working channel of the steerable instrument when in the stowed condition.

13. The steerable instrument according to claim 1 wherein the guide structure is dimensioned to fit through a working channel of the steerable instrument.

14. The steerable instrument according to claim 1 wherein deflection of the connector relative to the elongate body distal end provides position feedback of the guide structure relative to the elongate body.

15. The steerable instrument according to claim 1 wherein the position feedback of the guide structure relative to the elongate body is an input to an electronic motion controller used to control the steerable instrument.

16. The steerable instrument according to claim 1 wherein the deflection of a surface of the guide structure provides position feedback of the guide structure.

17. The steerable instrument of claim 1 further comprising a sensor on or in the guide structure that generates a signal when bent.

18. The steerable instrument of claim 17 wherein the signal generates an indication used to steer the elongate body.

19. The steerable instrument of claim 17 wherein the signal is used to provide position feedback of the guide structure.

20. The steerable instrument of claim 1 further comprising:
a roller on the guide structure.

21. The steerable instrument of claim 1 wherein the guide structure is an eversion tube.

22. The steerable instrument of claim 1 wherein the guide structure is adapted to be pneumatically advanced distally from the distal end of the elongate body distal end.

23. The steerable instrument of claim 22 wherein the connector is a tether.

24. The steerable instrument of claim 1 wherein the steerable instrument is a guide tube.

25. A method for advancing an instrument along a path, comprising:
providing an indication of the direction of the path of the instrument by movement of a guide structure in response to interaction with an object defining the path; and
adjusting the direction of the instrument based on the indication.

26. The method for advancing an instrument according to claim 25 wherein the adjusting step is performed by a user steering the instrument.

27. The method for advancing an instrument according to claim 25 wherein the adjusting step is performed by a control system that manipulates the instrument.

28. The method for advancing an instrument according to claim 25 further comprising:
looking through the guide structure to view the path.

29. The method for advancing an instrument according to claim 25 further comprising:
pneumatically advancing the guide structure along the path.

30. The method for advancing an instrument according to claim 25 wherein the indication of the direction of the path is provided by the movement of a connector attached to the guide structure.

31. The method for advancing an instrument according to claim 25 wherein the movement of the guide structure in response to interaction with an object defining the path is deflection of the surface of the guide structure.

32. The method for advancing an instrument according to claim 25 wherein indication of the direction of the path of the instrument is provided through interaction of an instrument in the guide structure with the path.

33. The method for advancing an instrument according to claim 25 wherein indication of the direction of the path of the instrument is provided by a sensor or instrument that generates a signal when bent.

34. The method for advancing an instrument according to claim 33 wherein the signal generated by the sensor provides bend information in two dimensions.
